(19) United States
(12) Plant Patent Application Publication
(10) Pub. No.: US 2004/0172731 P1
(43) Pub. Date: Sep. 2, 2004

(54) Hybrid tea rose plant named 'Zaryellow'

(76) Inventor: Keith Zary, Somis, CA (US)

Correspondence Address: CHRISTIE, PARKER & HALE, LLP, P.O. BOX 7068, PASADENA, CA, 91109-7068, US

(21) Appl. No.: 10/377,336

(22) Filed: Feb. 28, 2003

(51) Int. Cl. A01H 5/00

(52) U.S. Cl. PLT/134

(57) ABSTRACT

A hybrid tea rose plant variety producing large yellow flowers.

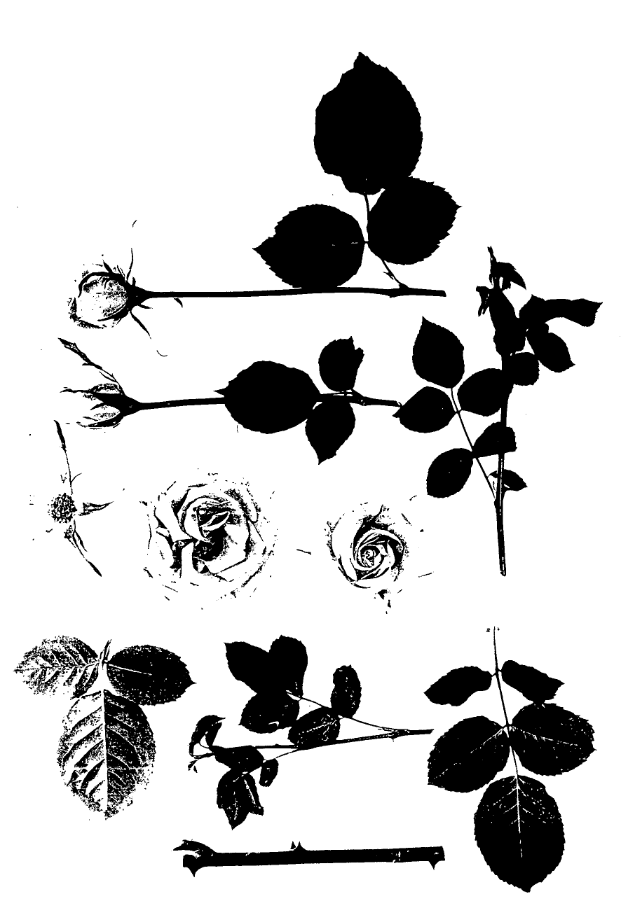

### BACKGROUND OF THE INVENTION

The present invention relates to a new and distinct hybrid tea rose variety which was originated by crossing as seed parent the variety known as ‘JACyem’ and as the pollen parent the variety known as ‘79-0004511’. The varietal denomination of this new rose is ‘ZARYELLOW’.

### SUMMARY OF THE INVENTION

Among the novel characteristics possessed by the new variety which distinguish it from its parents and all other varieties of which I am aware is its stable deep yellow color and larger flower size.

Asexual reproduction by budding of the new variety as performed in Somis, Calif., shows that the foregoing and other distinguishing characteristics come true to form and are established and transmitted through succeeding propagations. Other known forms of asexual reproduction may be used also.

### COMPARISON WITH PARENTS

‘Zaryellow’ has a deeper color than its seed parent and the color is stable throughout opening. The flower size of ‘Zaryellow’ is much larger than the flower size of the pollen parent.

### BRIEF DESCRIPTION OF ILLUSTRATION

The accompanying illustration shows typical specimens of the vegetative growth and flowers of the new variety in different stages of development and depicted in color as nearly true as it is reasonably possible to make the same in a color illustration of this character.

### DESCRIPTION OF THE NEW VARIETY

The following is a detailed description of the new variety, from specimens grown under glass in Richmond, Ind., in the month of January. The color terminology used is in accordance with The Royal Horticultural Society Colour Chart (RHSCC), and refers to plate numbers in the aforementioned color chart. Phenotypic expression may vary depending on light, environmental and cultural conditions.

### BUD

Peduncle: Long about 5¼ inch length, medium diameter, and erect with entirely smooth bark.

Color of bark: Near 144A.

Thorns: None.

Prickles: None.

Hairs: None.

Before calyx breaks:

Bud size.—Medium small.

Bud form.—Short pointed without a conspicuous neck; with foliaceous appendages on the surface of the bud and slender foliaceous parts extending beyond the tip of the bud equal to about ½ or more its length.

Color of calyx.—Near 144A, funnel shaped, some have a margin fringed with hairs and small appendages near 144A (ranging from about 1/16″ to ¼ inch long).

As calyx breaks/color: Near 17B shaded with 14C; base of petal near 145A.

As first petal breaks:

Bud size.—Medium large.

Bud form.—Long, pointed.

Color of petal.—Outside: Near 13B Inside: Near 14B.

Bud opening in greenhouse: Bud opens well.

### FLOWER

Flowers borne: One, on medium strong long stems.

Continuity in greenhouse: Continuous.

Qty of bloom in greenhouse: Moderate.

Bloom (open):

Size when fully open.—About 4½ inches to 5 inches.

Petalage: Double to 28 petals.

Form: At first high centered, becoming flat, remaining rolled outward at first; petals loosely roll outward at maturity.

Petals: Moderately thick, inside satiny, out side satiny.

Petals/Shape:

Outside petals.—Elliptic; apex mucronate.

Intermediate petals.—Elliptic; apex mucronate.

Inside petals.—Elliptic; apex mucronate.

The following description is of a newly opened flower from a rose grown in a greenhouse.

Color of outer petal:

Outside surface.—Near 17B.

Inside surface.—Near 14A.

Color of intermediate petal:

Outside surface.—Near 21B; base near 14B.

Inside surface.—Near 14A.

Color of inner petal:

Outside surface.—Near 21B; base near 14B.

Inside surface.—Near 14A.

Behavior of petals in greenhouse: Persist.

Flower longevity: About 7 days in February after cutting.

Color of open bloom: Near 14A.

Color three days open: Near 14A.

The following description was made from a rose that was open for three days in a greenhouse.

Color of outer petal:

Outside surface.—Near 13B.

Inside surface.—Near 13B shaded 13A.

Color of intermediate petal:

Outside surface.—Near 15A.

Inside surface.—Near 14A.

Color of inner petal:

Outside surface.—Near 16A.

Inside surface.—Near 13A.

### FOLIAGE

Leaves: Compound of three, five and seven leaflets, abundant, medium size, leathery.

Leaflets:

Shape.—Elliptical.

Apex.—Acute.

Base: Elliptical.

Margin: Simply serrate.

Color of leaflets:

Mature, upper surface.—Near 137A.

Mature, under surface.—Near 138B.

Young, upper surface.—Near 138A bordered 187A.

Young, under surface.—Near 181A shaded 181B.

Rachis:

Upper side.—Smooth, grooved, hairy with few hairs on the edges of the groves.

Under side.—Smooth, some have a few small thorns.

Stipules: Medium length ½ to ¾ inch, medium width 3/16 to ¼ inch with points which are medium length 3/16 turning out at an angle of more than 45 degrees.

### GROWTH

Habit: Bush, upright, branched.

Growth: Moderately vigorous.

Canes: Medium diameter.

Main stems: Near 146B.

Thorns.—Several, medium length, straight, with narrow base.

Color of thorns.—Near 166A shaded with 166B.

Prickles.—None.

Hairs.—None.

Branches: Near 146A.

Thorns.—Several, medium length, with straight narrow base.

Color of thorns.—Near 146B shaded 146C.

Prickles.—None.

Hairs.—None.

New shoots:

Color.—Near 184B shaded with 138B.

Thorns.—Few, medium length, straight with narrow base.

Color of thorns.—Near 46A; tip near 146C.

Prickles.—None.

Hairs.—None.

### REPRODUCTIVE ORGANS

Stamens: Medium number, arranged regularly about pistils.

Filaments: Short, medium length, most with anthers.

Color of filaments: Near 15A some with a base color 45B.

Color of anthers: Near 21A shaded 21B.

Pollen: Moderate.

Color of pollen: Near 162C.

Pistils: Many (Approx. 103).

Styles: Short, medium length, moderately thin, bunched.

Color of styles: Near 3D.

Color of stigma:

Ovularies: All encased in calyx.

Hips: N/A.

Sepals: Permanent, medium length, straight, funnel shaped; some have a margin fringed with hairs and appendages of about 1/16 to ¼ inch long; color near 144A.

Under side.—Near 143A; base 144C.

Upper side.—Near 138A; base near 138C.

Seeds: N/A.

I claim:

1. A new and distinct hybrid tea rose plant of the variety substantially as shown and described.
